(19) United States
(12) Patent Application Publication
(10) Pub. No.: US 2023/0075100 A1
(43) Pub. Date: Mar. 9, 2023

(54) ADVERSARIAL AUTOENCODER ARCHITECTURE FOR METHODS OF GRAPH TO SEQUENCE MODELS

(71) Applicant: INSILICO MEDICINE IP LIMITED, Hong Kong (HK)

(72) Inventors: Aleksandrs Zavoronkovs, Pak Shek Kok (HK); Evgeny Olegovich Putin, Saint Petersburg (RU); Kirill Sergeevich Kochetov, Saint Petersburg (RU)

(21) Appl. No.: 17/800,129

(22) PCT Filed: Feb. 19, 2021
(86) PCT No.: PCT/IB2021/051404

§ 371 Date: Aug. 16, 2022

Related U.S. Application Data
(60) Provisional application No. 62/978,721, filed on Feb. 19, 2020.

(51) Int. Cl. G06N 3/08

(52) U.S. Cl. CPC G06N 3/08

(57) ABSTRACT

A graph-to-sequence (G2S) architecture is configured to use graph data of objects to generate sequence data of new objects. The process can be used with objects types that can be represented as graph data and sequence data. For instance, such data is molecular data, where each molecule can be represented as molecular graph and in SMILES. Examples also include popular tasks in deep learning of image-to-text or/and image-to-speech translations. Images can be naturally represented as graphs, while text and speech can be natively represented as sequences. The G2S architecture can include a graph encoder and sample generator that produce latent data in a latent space, which latent data can be conditioned with properties of the object. The latent data is input into a discriminator to obtain real or fake objects, and input into a decoder for generating the sequence data of the new objects.

### CROSS-REFERENCE TO RELATED APPLICATIONS

This patent application claims priority to U.S. Provisional Application No. 62/978,721 filed Feb. 19, 2020, which provisional is incorporated herein by specific reference in its entirety.

### BACKGROUND

### Field

The present disclosure relates to an adversarial autoencoder architecture for methods of converting chemicals from one format to another, such as from a graph model to a sequence model.

### Description of Related Art

Deep neural networks (DNNs) are computer system architectures that have recently been created for complex data processing and artificial intelligence (AI). DNNs include machine learning models that employ more than one hidden layer of nonlinear computational units to predict outputs for a set of received inputs. DNNs can be provided in various configurations for various purposes, and continue to be developed to improve performance and predictive ability.

Deep learning has been used for a variety of purposes throughout its development, such as generating text from pictures or other functions. Recently, DNNs have been used for biomarker development, drug discovery and drug repurposing. In part, computer technology is being used in place of or to enhance standard drug discovery in order to offset the significant time and costs of identifying a potential drug and moving the potential drug through the regulatory process before it can be marketed as a commercial drug. While the standard drug discovery pipeline includes many stages, it is still a problem to find an initial set of molecules that may change the activity of a specific protein or a signaling pathway.

The hit rate of new drug candidates can be improved by removing compounds that do not show significant promise. Such compounds can be identified as unsuitable for further study at early stages with machine learning models, which can be used to estimate properties of the compound and guide the drug optimization process. Machine learning can be used to learn useful latent representations of molecules using variational autoencoders, graph convolutions, and graph message passing networks.

Artificial neural networks (ANNs) are a family of machine learning (ML) models, which are based on a concept of biological neurons that is broadly applied to diverse artificial intelligence tasks such as classification, regression, clustering, and object generation. Typically, a single artificial neuron takes so-called input signals (e.g., commonly represented as N-dimensional real-valued vectors) and outputs the sum of the input multiplied by the neuron's learnable weights to which some linear or nonlinear function, such as sigmoid or hyperbolic tangent, is applied. Usually, the ANN includes a large number of artificial neurons that are organized layer by layer. Each ANN has input layers, hidden layers, and output layers. The DNNs are ANNs with one or more hidden layers.

Since almost all tasks in ML are formulated in terms of optimization problems, each DNN has certain training and validation procedures that are based on a backpropagation algorithm. For example, in the case of binary classification on a training stage, some loss function (e.g., binary cross entropy) is computed with respect to training samples (e.g., samples for which true label is available), and then the aggregated errors are backpropagated to the DNN input layer. This process is usually repeated multiple times until the protocol converges on the model. The validation stage trained DNN predicts labels for unseen objects (e.g., model does not see objects during training) and some quality metric is computed to estimate the efficacy of a trained DNN model.

In some instances, it is beneficial to represent complex high-dimensional objects in simpler form and in lower dimensional space. Accordingly, there exists a specific DNN called an autoencoder (AE). The AE includes two DNNs: an encoder and a decoder. The encoder compresses input signals into a low dimensional space called the latent representation. The decoder takes the latent representation of the input objects and returns reconstructed input signals. The training objective for the AE is to minimize the error between input signals and reconstructed ones.

The generative adversarial network (GAN) is a type of DNN that is based on paradigm adversarial learning, and is capable of generating realistic objects, such as images, texts, speech, and molecules as well as others. In this concept, there is a minimax comparison between two players represented as DNNs, which are a generator and a discriminator. The generator takes some of the sample data (e.g., typically sample data from a standard normal distribution or uniform distribution of the original object data) and produces fake samples. The discriminator takes a sample and decides whether or not this sample is drawn from a real distribution (e.g., comes from a real training set) or from the fake sample that is produced by the generator. The generator and discriminator compete against each other, and it is proved that such a minimax comparison has a Nash equilibrium. Both the generator and discriminator are trained via backpropagation, and the error of the one is the payoff of the other. GAN can be easily extended for conditional generation.

An adversarial autoencoder (AAE) is a GAN-based AE model. It has three DNN components: an encoder, a decoder, and a discriminator. In the AAE, the encoder is the same as a generator, and thereby the encoder serves for two purposes: 1) it compresses objects into the latent space as with the encoder; 2) it receives sample data (e.g., usually from a standard normal distribution of the original object data or other training data of objects) and outputs fake samples (e.g., of objects) in latent space as with the generator. As usual, the decoder maps points of the latent space into objects. A distinct difference between AAE and GAN architectures is that in the AAE the discriminator classifies not objects but their latent representation (e.g., obtained by using the encoder).

The architecture where the encoder and generator are not the same is called an adversarial regularized autoencoder (ARAE). In case of the ARAE, there are no restrictions on latent space that are in the AAE, in part because the explicit generator can induce any distribution in the latent space. Therefore, the ARAE is more flexible than the AAE.

The DNN referred to as Sequence-to-Sequence (Seq2Seq) is a special case of AE architecture where both the encoder and decoder are recurrent neural networks (RNNs). In the case of Seq2Seq, the input of the encoder and output of the decoder are symbolic sequences.

The DNN referred to as Graph-to-Sequence (G2S) is a conditional AAE/ARAE model that receives objects in a graph representation and then outputs objects in a sequence or string representation. Some aspects of the G2S is to keep up structural and topological information of objects by using graph representation. G2S encoder compresses graphs to latent points preserving their structural relationships, then G2S decoder maps latent points into sequences or strings. Thus, the G2S model can be useful in a number of instances. However, the G2S modeling can still be improved.

Therefore, it would be advantageous to improve a G2S model in instances where the object is a complex graph (e.g., molecule) that can be expressed as a sequence (e.g., SMILES).

### SUMMARY

In some embodiments, a computer-implemented method for training a model to generate an object can have an autoencoder step comprising: providing a variational, adversarial or combination of variational and adversarial autoencoder architecture configured as a graph-to-sequence (G2S) model; inputting graph data for a plurality of real objects into an encoder of the G2S model; generating sequence data from latent space data with a decoder of the G2S model; generating discriminator output data from a discriminator of the G2S model; performing an optimization for the encoder and decoder; and reporting a trained G2S model.

In some embodiments, a computer-implemented method for training a model to generate an object can include an autoencoder step, such as follows: providing an adversarial autoencoder architecture configured as a graph-to-sequence (G2S) model; obtaining graph data for a plurality of real objects; inputting the graph data into an encoder; generating latent data having latent vectors in a latent space from the graph data with the encoder; obtaining property data of the real objects; concatenating the latent vectors from the graph data with the property data in the latent space; inputting latent space data into a decoder; generating sequence data from the latent space data with the decoder, wherein the sequence data represents real objects and includes symbol logits; computing a log-likelihood between the logits of the sequence data and sequence data of the obtained graph data; inputting latent space data into a discriminator; generating discriminator output data from the discriminator, wherein the discriminator output data includes discriminator logits; computing a log-likelihood of the discriminator logits and labels “1”, wherein labels “1” is a real output data of the discriminator; performing a gradient descent step for the encoder and decoder; and reporting a trained G2S model. The reporting can be via physical report (e.g., paper) or electronic report, which may be displayed on a display screen of a computing system, or the reporting can store the model in a database.

In some embodiments, a computer-implemented training protocol can include a generator step comprising: inputting the sample data of a normal distribution into a generator of the G2S model; generating discriminator sample data with the discriminator; performing an optimization for the generator; and reporting a generator trained G2S model.

In some embodiments, computer-implemented a method for training a model to generate an object can include a generator step comprising: obtaining sample data of a normal distribution of object data; inputting the sample data into a generator; generating sample latent vectors with the generator, wherein the sample latent vectors are in the latent space; concatenating the property data with the sample latent vectors; inputting latent space data into the discriminator to obtain discriminator sample data having sample logits; computing a log-likelihood of the discriminator sample logits and labels “1”, wherein labels “1” is a real output data of the discriminator; computing a Jacobian clamping term for the generator; performing a gradient descent step for the encoder and decoder; and reporting a generator trained G2S model. The reporting can be via physical report (e.g., paper) or electronic report, which may be displayed on a display screen of a computing system, or the reporting can store the model in a database.

In some embodiments, the computer-implemented training can include a discriminator step comprising: computing an effectiveness of the discriminator; performing an optimization for the discriminator using the computed effectiveness; and reporting a discriminator trained G2S model.

In some embodiments, a computer-implemented method for training a model to generate an object can include a discriminator step comprising: computing a log-likelihood of the discriminator sample logits and labels “0”, wherein labels “0” is a fake output data of the discriminator; performing a gradient descent step for the discriminator using outcome from the log-likelihood of the discriminator logits and labels “1”, and from log-likelihood of the discriminator sample logits and labels “0”; and reporting a generator trained G2S model. The reporting can be via physical report (e.g., paper) or electronic report, which may be displayed on a display screen of a computing system, or the reporting can store the model in a database.

In some embodiments, a computer-implemented method of generating a new object can include: providing a graph-to-sequence (G2S) model, such as described herein; inputting graph data of real objects and properties thereof into the G2S model; training the G2S model with the graph data and property data to obtain a trained G2S model; inputting desired property data of a desired property into the trained G2S model; generating a new object with the desired property with the trained G2S model; and reporting the new object that has the desired property. In some aspects, the method (e.g., non-computer implemented steps) can include: creating a real version of the new object (e.g., physical object with properties); and validating the new object to have the desired property. In some aspects, the real object is a molecule and the property of the molecule includes biochemical properties and/or structural properties. In some aspects, the real objects are images and the properties are descriptions having sequences of natural language words.

In some embodiments, the computer-implemented methods of generating the new object can include: inputting sample data of a normal distribution into the generator of the G2S mode; conditioning latent vector data in the latent space with at least one desired property of the object; inputting conditioned latent vector data into the decoder; and generating sequence data of a generated object having the at least one desired property. In some aspects, the normal distribution is a normal distribution of real objects having the at least one desired property.

In some embodiments, one or more non-transitory computer readable media are provided that store instructions that in response to being executed by one or more processors, cause a computer system to perform the operations of any of the computer-implemented methods recited herein.

In some embodiments, a computer system can include: one or more processors; and one or more non-transitory computer readable media storing instructions that in response to being executed by the one or more processors, cause the computer system to perform operations of any of the computer-implemented methods.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE FIGURES

The foregoing and following information as well as other features of this disclosure will become more fully apparent from the following description and appended claims, taken in conjunction with the accompanying drawings. Understanding that these drawings depict only several embodiments in accordance with the disclosure and are, therefore, not to be considered limiting of its scope, the disclosure will be described with additional specificity and detail through use of the accompanying drawings.

FIG. 1 illustrates a schematic representation of the graph-to-sequence (G2S) model architecture.

FIG. 2 includes a flowchart that illustrates a training process for a G2S model.

FIG. 3 includes a flowchart that illustrates a process for generating an object with a trained G2S model.

FIG. 4 includes a graph that shows an example of a Tanimoto similarity with target molecule maximization using a G2S model with REINFORCE optimization.

FIG. 5 includes a graph that shows an example of quantitative estimation of drug likeness (QED) maximization using a Bayesian optimization algorithm on G2S latent space.

FIG. 6 illustrates a schematic representation of a modified G2S model architecture for molecule generation based on scaffold and/or fragment conditioning.

FIG. 7 illustrates examples of scaffolds and the resulting generated molecules with given scaffolds based on the modified G2S model architecture of FIG. 6.

FIG. 8 illustrates a schematic representation of a computing system that can be used in the methods described herein.

The elements and components in the figures can be arranged in accordance with at least one of the embodiments described herein, and which arrangement may be modified in accordance with the disclosure provided herein by one of ordinary skill in the art.

### DETAILED DESCRIPTION

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

Generally, the present technology includes an improved Graph-to-Sequence (G2S) model and protocol for improving the G2S output. The G2S can utilize graph data as input into an encoder, such as described herein. The graph data can be based on datasets such as social networks, citation networks, molecular structures, or others. The graph structured data can be present with various sizes of unordered nodes and each node in a graph can have a different number of neighbor nodes. Graph structured data is known and can be obtained through various techniques depending on the source data. Some examples include an adjacency matrix, feature matrix, or others. Accordingly, the G2S can be used with any source data that can be converted into graph structured data, or the source data can be graph structured data. For example, the source data can be a sequence data for a molecule, such as simplified molecular-input line-entry system (SMILES), that can be converted into graph structured data by known techniques. Then, the graph structured data of the molecules can be input into the encoder, such as described herein.

Accordingly, the present G2S model can be used for generating new chemical entities, but can also be used for generating other objects that can be represented in both graph structured data (e.g., graph data) and sequence structured data (e.g., sequence data). The data can be obtained in either graph data or sequence data. When sequence data is obtained as source data, a transformation is performed to convert the sequence data to the graph data. A transformation function can be tailored so that it depends on the type of the input data. An example of a transformation protocol for SMILES is as follows: each molecule can be represented as SMILES and molecular graph, so the transformation process from sequence to graph is just replacing the representation of the molecule from sequence representation (e.g., SMILES) to the graph representation (e.g., molecular graph). The graph representation can often include an adjacency matrix (e.g., connections between atoms) and node (e.g., atoms) features.

In some embodiments, the data can be configured to improve correctness of G2S model training and validation. Accordingly, the data can have the following properties with respect to graph data and sequence data. First, each sample (or composed sample) of data should be able to be represented as a graph and as a sequence. Second, the data should allow for mapping of the samples from graph representation to sequence representation, and vice versa. In an example, such data is molecular data, where each molecule can be represented as a molecular graph and in a molecular sequence, such as SMILES format.

Additionally, popular tasks in deep learning can use image-to-text and/or image-to-speech translations. Images can be naturally represented as graphs, while text and speech can be natively represented as sequences. Accordingly, these types of data, as well as others, can be represented as graph or sequence data, and used in the G2S model training and validation protocols.

In some embodiments, the G2S protocol described herein can be applied on molecular datasets. For example, the G2S protocol can been applied to the QM9 dataset containing small organic molecules with up to nine heavy atoms, and applied to the ZINC250 dataset from the ZINC database containing commercially available compounds (molecules) that could be possible drugs. These molecules can be used for virtual screening. For all molecules from the QM9 and ZINC250 datasets, several chemical properties were calculated, including quantitative estimation of drug likeness (QED), LogP (measure of lipophilicity) and other molecular descriptors, which can be used as properties for conditional generation in the G2S model. The data may be obtained as sequence data and then transformed to the graph data for use in the G2S model. The graph data and the properties of each example molecule then can be used.

The G2S models may have various configurations. However, the present G2S model described herein can provide an improvement of molecule generation by including complexities of graph data, which can be used to obtain more accurate sequence data of a generated molecular object.

In some embodiments, a G2S model can include an encoder, decoder, generator, and discriminator. In some aspects, the G2S model is trained as an ARAE. In some aspects, the G2S model is trained in AAE manner. In some aspects, the encoder is a DNN, which can be configured as: multilayer perceptron (MLP); convolutional neural network (CNN) and its variants (e.g., like diagonal CNN); any kind of graph convolutional networks (GCNs); or any kind of graph neural network (GNNs). The DNNs that can be used are configured to process graph structure objects (e.g., molecules, proteins, computer viruses, etc.) and output latent vectors corresponding to the input graph data. In some aspects, the decoder is a DNN, which can be configured as: MLP; long short term memory network (LSTM); or gated recurrent unit network (GRU). The decoder is configured to output string sequences using latent vectors. In some aspects, the discriminator is a DNN, such as a 1D CNN or MLP, which takes latent points and outputs binary labels classifying points into real or fake categories. In some aspects, the generator is a DNN, such as 1D CNN or MLP, which receives sample from a standard normal distribution and outputs points in latent space.

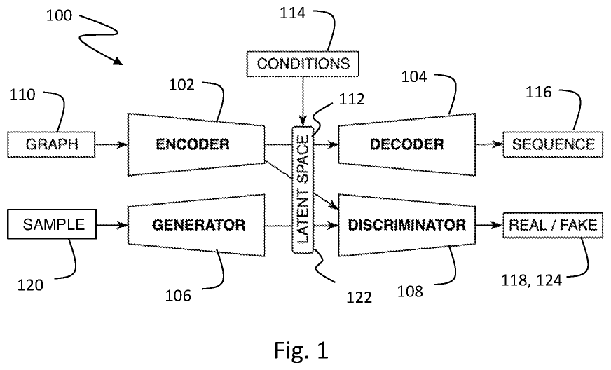

FIG. 1 illustrates an embodiment of a G2S architecture 100, which is shown to include the encoder 102, decoder, 104, generator 106, and discriminator 108. As shown, the graph data 110 is provided to the encoder 102, which processes the graph data 110 to obtain the latent space data 112. The G2S architecture 100 also includes the conditions data 114 of the objects (e.g., molecules) in the graph data 110 being linked to the latent space data 112, where the conditions data 114 is concatenated with latent vectors of the objects in the latent space data 112, such as in the latent space. The conditions data 114 can include property data for each object, and the property data is linked with the latent vectors of the respective object (e.g., in cases of conditional generation). The latent space data 112 from the encoder 102, optionally concatenated with the conditions data 114, can be provided to the decoder 104, which is processed to obtain the sequence data 116. The sequence data 116 can include symbol logits, which is obtained from the decoder 104. As described in more detail herein, a log-likelihood between the symbol logits (e.g., from the decoder 104 in the sequence data 116) can be computed with the sequence data that corresponds with the graph data 110 that was introduced into the encoder 102. In some instances, the sequence data that corresponds with the graph data 110 can be obtained, and in others, the graph data 110 is obtained from the sequence data. In any event, this sequence data that corresponds with the graph data 110 is compared with the sequence data 116 that is output from the decoder 104, which comparison can be via the computation of a log-likelihood between the symbol logits of the sequence data 116 and of the sequences data that corresponds with the graph data 110.

The latent space data 112 from the encoder 102, optionally concatenated with the conditions data 110, can be provided to the discriminator 108 and the discriminator 108 can generate output data 118. The output data 118 can be real output or fake output, which is described more below. Then, the output data 118 can include symbol logits, which is obtained from the discriminator 108. As described in more detail herein, a log-likelihood between the symbol logits (e.g., from the discriminator 108 in the output data 118) can be computed with labels “1.” The discriminator 108 can output labels “1” for real output that includes objects that match the objects of the graph data 110, and can output labels “0” for fake output that only includes synthetic objects. Accordingly, the output data 118 can be sequence data that can correspond with the sequence data of the graph data 110 that was introduced into the encoder 102 (e.g., which can be real or “1”). In any event, this sequence data that corresponds with the graph data 110 is compared with the sequence data of the output data 118 that is output from the discriminator 108, which comparison can be via the computation of a log-likelihood between the symbol logits of the output data 118 and of the sequences data that corresponds with the graph data 110 that are real or have the label “1.”

In some instances, a gradient descent step calculation can be performed for the encoder 102 and the decoder 104 using the losses that are calculated (e.g., the log-likelihood) between logits and sequences from the decoder 104 and between logits and the labels “1.” Sequences or objects with lower losses can be preferred. The gradient descent step calculation can be performed until the losses are lower than a loss threshold.

Additionally, the G2S architecture 100 includes the generator 106 that is configured for receiving sample data 120. The sample data 120 can be the sample from a natural standard distribution of the object data. The generator 105 can then generate sample latent space data 122 (e.g., which is different from the latent space data 112 from the encoder 102 and the graph data 110, e.g., graph latent space data 112) in the latent space. The G2S architecture 100 also includes the conditions data 114 of the objects (e.g., molecules) in the graph data 110 being linked to the sample latent space data 122, where the conditions data 114 is concatenated with latent vectors of the objects in the latent space data 122. The conditions data 114 can include property data for each object, and the property data is linked with the latent vectors of the respective object (e.g., in cases of conditional generation). The latent space data 122 from the generator 106, optionally concatenated with the conditions data 1104, can be provided to the discriminator 108, which is processed to obtain the sample output data 124. The sample output data 124 can be real output or fake output, which is described more below. Then, the sample output data 124 can include symbol logits, which is obtained from the discriminator 108. As described in more detail herein, a log-likelihood between the symbol logits (e.g., from the discriminator 108 in the sample output data 124) can be computed with labels “1.” The discriminator 108 can output labels “1” for real output that includes objects that match the objects of the sample data 120, and can output labels “0” for fake output that only includes synthetic objects. Accordingly, the output data 124 can be sequence data that can correspond with the sequence data of the sample data 120 that was introduced into the generator 106 (e.g., which can be real or “1”). In any event, this sequence data that corresponds with the sample data 120 is compared with the sequence data of the output data 118 that is output from the discriminator 108, which comparison can be via the computation of a log-likelihood between the symbol logits of the output data 124 and of the sequences data that corresponds with the sample data 120 that are real or have the label “1.”

In some embodiments, the G2S architecture 100 can compute a Jacobian clamping term for the generator. To make the latent space smoother, Jacobian clamping (JC) regularization can be performed. The function of the JC is to clamp a Jacobian norm of the generator 106 between two values. In other words, the JC goal is to minimize the absolute difference of perturbations between the sample data 120 inputs of the generator 106 and outputs (e.g., latent space data 122) of the generator 106 that are produced using these inputs. The JC is a regularization term added to the common model loss.

In some instances, a gradient descent step calculation can be performed for the generator 106 and the discriminator 108 using the losses that are calculated (e.g., the log-likelihood) between logits and the labels “1” and with the outcome of the Jacobian clamping. Sequences or objects with lower losses can be preferred. The gradient descent step calculation can be performed until the losses are lower than a loss threshold.

In some embodiments, the G2S architecture 100 can compute a log-likelihood between the logits (e.g., from the discriminator 108 in the sample output data 124) can be computed with labels “0” (fake). The discriminator 108 can output labels “0” for fake output that only includes synthetic objects. Accordingly, the output data 124 can be sequence data that can correspond with the sequence data of the sample data 120 that was introduced into the generator 106. Also, a gradient descent step calculation can be performed for the discriminator 108 using the losses that are calculated (e.g., the log-likelihood) between logits and the labels “0” and with the loses (e.g., the log-likelihood) from the discriminator 108 in the output data 118 and labels “1” described above. Sequences or objects with lower losses can be preferred. The gradient descent step calculation can be performed until the losses are lower than a loss threshold.

In some instances, the losses are not sufficiently small. As a result, the rate of learning of the autoencoder can be decreased. The protocol with the G2S architecture 100 can be performed with iterations until the loss is suitable.

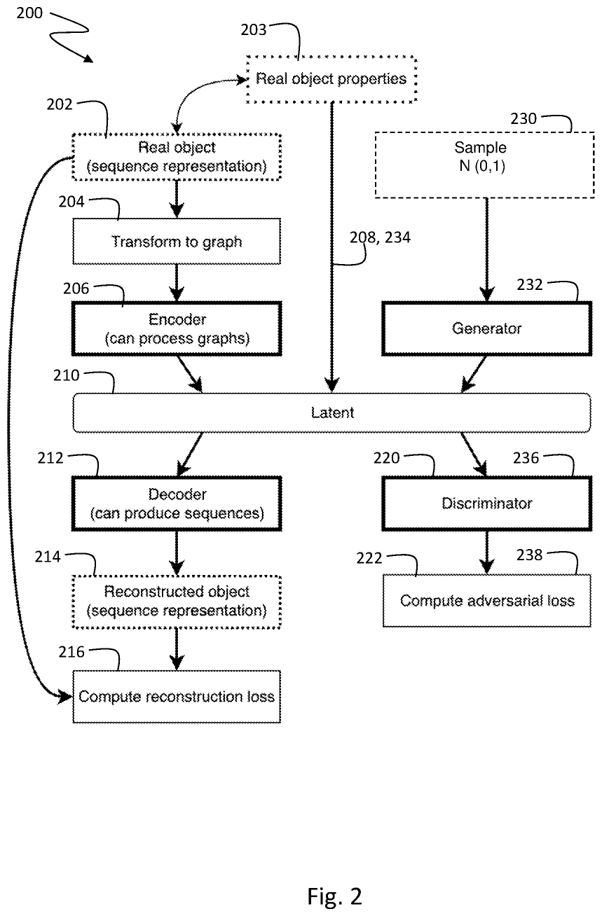

FIG. 2 illustrates a flowchart for a model training process 200 that can be used with the G2S architecture 100 of FIG. 1. The model training process can be performed as described. The model training process can include an autoencoder step as described below. The model training process 200 can include obtaining a real object data, which can be in the form of a sequence representation of that real object at block 202. The real object can have real object properties 203 that are relevant to the real object, which object properties can vary as desired for the generation of objects that match the real object in some way. The real object may be associated with the real object properties. For example, a minibatch of real objects that are represented as sequence data can be sampled along with the properties of the real object. In some aspects, the real object is obtained in a sequence representation, and then the model training process 200 can include a transformation protocol that transforms the sequence representation of the real object to the graph representation of the real object at block 204. That is, the sequence data for the real object is transformed to graph data, which can be by any transformation protocol. However, the graph data for the real object can be directly obtained without having to perform the transformation, but then the graph data may need to be converted to original sequence data so that the sequence data output from the decoder can be compared with the original sequence data. The graph data of the real object can then be provided as input into an encoder, which is configured to process graph data into latent vectors in the latent space, at block 206. Accordingly, the encoder can obtain the latent vectors of the graph data. The model training process 200 can concatenate (e.g., link) the real object properties 203 with the latent vectors at arrow 208. The concatenation can be helpful when there is a case of conditional generation of latent vectors by the encoder. Accordingly, the latent space 210 can include the latent vectors of the real object associated with the real object properties.

The data in the latent space 210, whether the latent vectors with or without being concatenated with the real object properties, can be input into the decoder and processed to obtain sequence data at block 212. That is, the decoder can produce a reconstructed object 214, such as a sequence representation of the reconstructed object. The log-likelihood between logits from block 212 (e.g., the reconstructed object 214) and sequences from block 202 can then be computed, such as described herein.

The reconstructed object can then be compared with the real object, and the reconstruction loss can be computed at block 216.

Additionally, the model training process 200 can include inputting the latent data from the latent space 210 into the discriminator to obtain discriminator output data at block 220. Then the adversarial loss of the discriminator output data can be computed at block 222. In some aspects, computing the adversarial loss can include computing the log-likelihood between logits from block 220 and labels ‘1’ (e.g., real). Then, the process 200 can include performing the gradient descent step for encoder and decoder using losses from block 216 and the losses of the computed log-likelihood between logits from block 220 and labels ‘1’ (e.g., real).

FIG. 2 also shows that the model training process 200 can include a generator step. Accordingly, a minibatch of sample data (e.g., N(0, 1) of normal distribution) can be sampled at block 230. The sample data can be input into the generator for processing to obtain latent vectors of sample data at block 232. The latent vectors of sample data can be in the latent space 210. The model training process 200 can concatenate the real object properties 203 with the sample latent vectors at arrow 234. The concatenation can be helpful when there is a case of conditional generation of latent vectors of sample data by the generator. Accordingly, the latent space 210 can include the latent vectors of the sample data associated with the real object properties.

The data of the latent vectors of sample data, with or without being associated with the real object properties, in the latent space 210 can be input into the discriminator to obtain discriminator outputs of the sample data latent vectors at block 236. The discriminator outputs of the sample data latent vectors can then be used to compute the adversarial loss at block 238.

Then, a log-likelihood between the symbol logits (e.g., from the discriminator in the sample output data) can be computed with labels “1.” The discriminator can output labels “1” for real output that includes objects that match the objects of the sample output data, and can output labels “0” for fake output that only includes synthetic objects. Accordingly, the sample output data can be sequence data that can correspond with the sequence data of the sample data that was introduced into the generator (e.g., which can be real or “1”). In any event, this sequence data that corresponds with the sample data is compared with the sequence data of the output data that is output from the discriminator with the sample vector data, which comparison can be via the computation of a log-likelihood between the symbol logits of the sample output data and of the sequences data that corresponds with the input sample data that are real or have the label “1.”

In some embodiments, the model training process 200 can compute a Jacobian clamping term for the generator. To make the latent space smoother, Jacobian clamping (JC) regularization can be performed. In some instances, a gradient descent step calculation can be performed using the losses that are calculated (e.g., the log-likelihood) between logits and the labels “1” and with the outcome of the Jacobian clamping. Sequences or objects with lower losses can be preferred. The gradient descent step calculation can be performed until the losses are lower than a loss threshold.

FIG. 2 also shows that the model training process 200 can include a discriminator step. As such, the discriminator step can compute a log-likelihood between the logits (e.g., from the discriminator in the sample output data) can be computed with labels “0” (fake). The discriminator can output labels “0” for fake output that only includes synthetic objects. Accordingly, the output sample data can be sequence data that can correspond with the sequence data of the sample data 120 was introduced into the generator. Then, a gradient descent step calculation can be performed for the discriminator using the losses that are calculated (e.g., the log-likelihood) between logits and the labels “0” and with the loses (e.g., the log-likelihood) from the discriminator in the output data and labels “1” described above. Sequences or objects with lower losses can be preferred. The gradient descent step calculation can be performed until the losses are lower than a loss threshold. In some instances, the losses are not sufficiently small. As a result, the rate of learning of the autoencoder can be decreased. The protocol with the G2S architecture 100 can be performed with iterations until the loss is suitable.

The following example can be used as a training procedure for the G2S model (FIG. 1). The training procedure was performed with a stochastic gradient descent using Adam optimizer with an initial learning rate equal to 0.001 for autoencoder and 0.0001 for generator and discriminator. For each iteration, the following steps are performed (FIG. 2): an autoencoder step; a generator step, a discriminator step, and optionally, a decrease rate for autoencoder step.

The autoencoder step can be performed as follows: a) Sample minibatch of real objects represented as sequences with their properties; b) Transform sampled real objects into graphs; c) Obtain latent vectors of graphs using encoder; d) Concatenate properties with latent vectors in case of conditional generation; e) Obtain sequences with symbol logits (wikipedia.org/wiki/Logit) using the decoder; f) Compute log-likelihood between logits from Step e) and sequences from Step a); g) Obtain outputs of discriminator using latent vectors of graphs from Step c); h) Compute log-likelihood between logits from Step g) and labels ‘1’ (e.g., real); and i) Perform the gradient descent step for encoder and decoder using losses from Step f) and Step h).

The generator step can be performed as follows: a) Sample minibatch of object data to get sample data of the distribution N (0, 1); b) Obtain latent vectors of sample data using generator; c) Concatenate properties with latent vectors in case of conditional generation; d) Obtain outputs of discriminator using latent vectors from Step c); e) Compute log-likelihood between logits from Step d) and labels ‘1’ (e.g., real); f) Compute Jacobian clamping term for the generator; and g) Perform the gradient descent step for generator using losses from data obtained in Step e) and Step f).

The discriminator step can be performed as follows: a) Compute log-likelihood between logits from Generator Step d) and labels ‘0’ (e.g., fake); and b) Perform the gradient descent step for discriminator using losses from Autoencoder Step h) and Discriminator Step a).

Then, if needed or desired, the learning rate for the autoencoder step can be decreased. Then the protocols can be performed again, and subsequent iterations can be performed until the loss is minimized or the outcome is suitable.

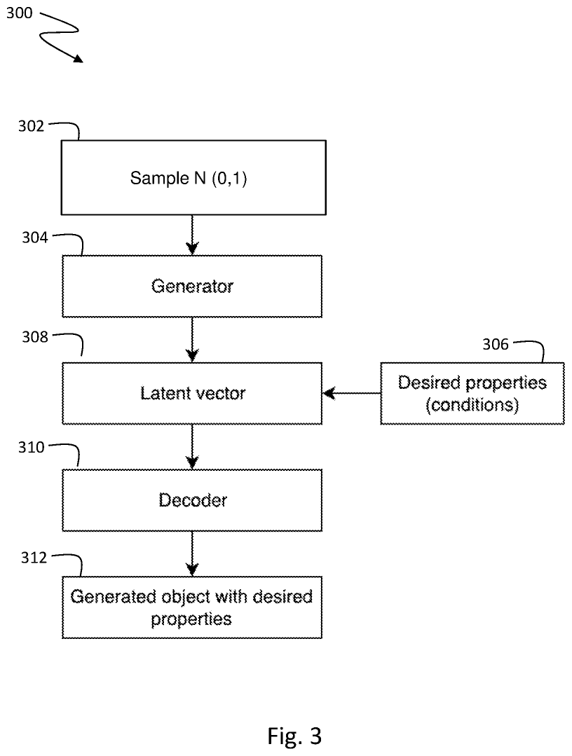

FIG. 3 illustrates a method of generating objects 300, where the objects are generated with predefined desired properties. Generally, the method 300 uses a G2S model that has been trained as described herein. Once the G2S model is trained, the objects can be generated. The method can include sampling the object data to get sample data (e.g., N(0, 1)) at block 302. Then, the method 300 can include inputting sampled data into the generator to produce sample latent vectors at block 304. The desired properties (e.g., generative conditions) for the objects to be generated by the decoder are provided at block 306. The sample latent vectors are then concatenated with the desired properties (e.g., generative conditions) to obtain a concatenated presentation of the sample latent vectors at block 308. The concatenated representations of the sample latent vectors are input into the decoder at block 310. The decoder then takes the concatenated representations of the sample latent vectors and produces the sequence data at block 312. The sequence data is data of the object that has the desired properties (e.g., generative conditions). For example, the sequence data can be SMILES sequences when the object is a molecule. The desired properties can provide guidance as to the object that is generated that has the sequence data. Accordingly, during generation, the properties need to be concatenated with the latent vector (produced by generator). Then, the decoder produces the SMILES sequences using the final latent vectors with the properties.

In some embodiments, the generation of objects can be done with optimization of properties. The generation of objects can be performed during or after a training protocol, such as described herein. There are several different techniques for performing property optimization protocols, which can be used jointly with the G2S model to generate objects with the desired properties. As such, the objects can be optimized to have certain properties that are associated with the objects. The desired properties can be identified and concatenated with the latent vectors as described herein, and the result is objects being generated that are optimized with the desired properties. For example, during training the optimization reinforcement learning was performed in combination with the G2S model. For example, after training optimization, Bayesian optimization and generative topographic mapping were tested.

Accordingly, the methods of training can be supplemented with reinforced learning protocols. In some embodiments, the reinforced learning protocols utilize the REINFORCE algorithm in combination with the G2S model in order to find more molecules with desired properties. The reinforcement protocol can use rewards that can steer the generated molecules toward those with the desired properties, and thereby more molecules can be generated with the desired properties. In particular, during the training stage the G2S model can use conditional generation (e.g., condition is a real valued vector with desired property that is directly passed to latent space of the model), and use reinforcement learning techniques, such as REINFORCE or others. However, when the G2S model is trained, it is feasible to use latent manifold in order to find areas covering objects with desired properties, methods to do so are Bayesian optimization and generative topographic mapping, which both can be used in combination with trained G2S. In some aspects, the reinforced learning is used in different G2S variations.

In some embodiments, the REINFORCE is a family of reinforcement learning methods which directly update the policy weights through the following rule:

Δθₜ=α∇θlogπθ(aₜ|sₜ)vₜ;

wherein α is the learning rate, πθ(aₜ|sₜ) is the policy (mapping actions to probabilities), and vₜ is a sample of the value function at time t collected from experience.

In some embodiments, the reinforced learning uses policy gradient methods, which can include a family of reinforced learning approaches that based on optimization of the policy by using gradient descent. The reinforced learning can be are used in combination with G2S in order to find more molecules with predefined desired properties (rewards), such as described above. In some aspects, the REINFORCE algorithm has the following update rule:

∇_{θ}J(θ)=Σₜ₌₀^{T-1}(Gₜ−bₜ)∇_{θ}logπ_{θ}(aₜ|sₜ);

wherein J is the objective function, T is the length of output sequence, π_{θ}(aₜ|sₜ) is the policy (e.g., mapping from states to probability distribution over actions), Gₜ is a discounted reward and bₜ is baselined return.

In some embodiments, the methods of reinforcement learning can be implemented during training, such as in one of the training methods described herein. The reinforcement can be performed after pretraining. After pretraining the G2S model on an original dataset, all G2S model parts and parameters were set (e.g., held or frozen) except for the generator and decoder. After that setting of the G2S model, the training procedure with reinforcement can be performed as follows: 1) Sample batch of object data to get sample data of distribution N(0, 1); 2) Obtain latent vectors using the generator; 3) Obtain objects using the decoder; 4) Calculate properties (e.g., rewards) of generated objects; 5) If the reward for some of the generated objects are close enough with the desired reward, the parameters of the generator and decoder change in order to better explore the latent manifold of corresponding good rewarded objects; and 6) Repeat Steps 1 through 5) until convergence.

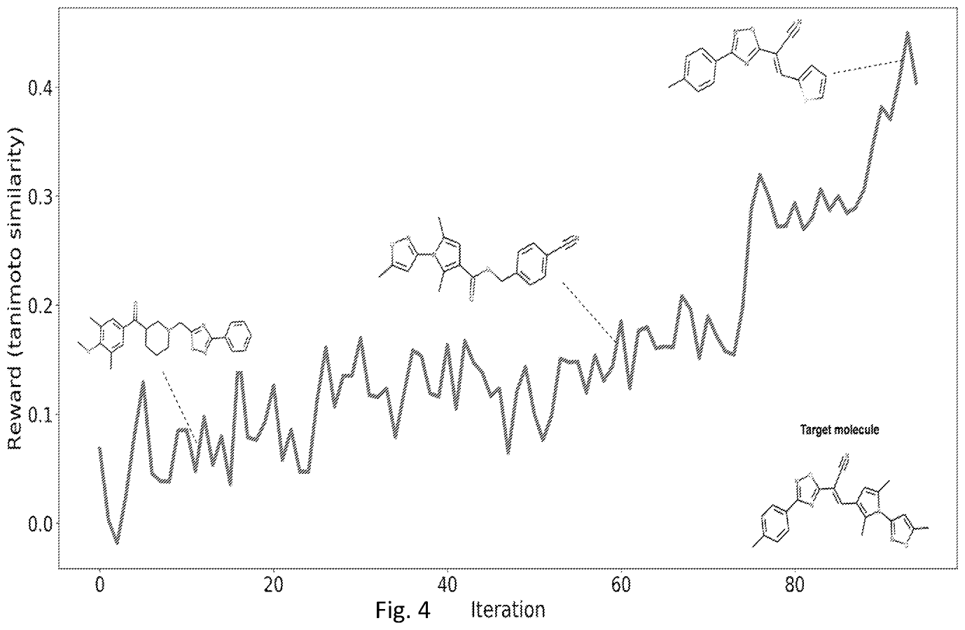

In some embodiments, the rewards (E.g., which can be properties of the object) that are used in combination with G2S model can include: solubility; LogP; SLogP; QED, and Tanimoto similarity with target molecule. An example of convergence of the G2S and REINFORCE model is presented on FIG. 4, using Tanimoto similarity.

In some embodiments, the latent space having the latent vectors can be optimized, such as by Bayesian optimization (BO). Accordingly, the methods recited herein may also include a step of performing a BO protocol. The BO of the latent space can be used in combination with a previously trained G2S model in order to determine or identify latent space manifolds with desired properties. The BO protocol can be performed such that the function builds a probability model of the objective function, such as a reward function. The protocol can use the probability model to select the most promising areas (e.g., objects, objects in a certain region, manifold, etc.) from the latent space of the G2S model. These selected promising areas can then be evaluated with a true objective function in order to identify one or more objects, such as of the generated sequence data. In some aspects, the BO protocol can include the following protocol: 1) Initiate a surrogate model (e.g., a regression model, such as linear regression model); 2) Sample a batch of points from the most promising areas of the latent space of the trained G2S in terms of desired properties for the objects; 3) Obtain the objects using decoder; 4) Calculate properties of generated objects; 5) Update the surrogate model with the sampled batch from Step 2) and real properties from Step 4); and Repeat Steps 2) through 5) until convergence (or Steps 1) through 5)).

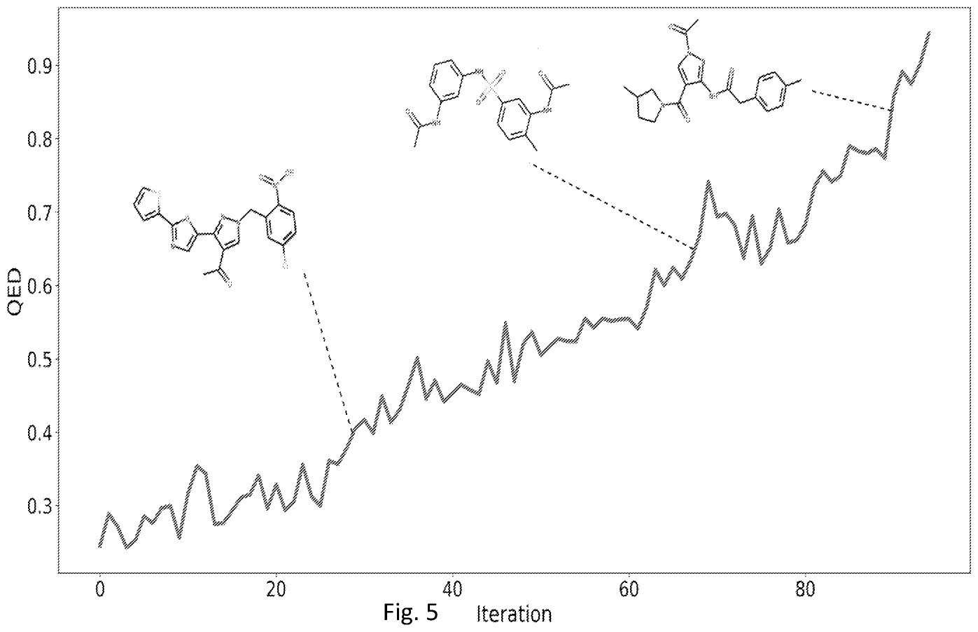

The BO protocol can be performed with one or more desired properties for the objects. As such, certain properties may be preferred over other properties, or a property hierarchy may exist, which can be used during the BO protocol. Accordingly, the BO protocol can be performed with preferred properties so that the optimization optimizes those preferred properties in the generated objects. There are a lot of properties to optimize during molecule generation process, such as those described herein or others, where any property of an object may be used. For molecule objects, the properties can be any chemical property from structural requirements to physical-chemical properties. For example, the process of QED optimization using the trained G2S model and BO protocol is presented on FIG. 5.

In some embodiments, the latent space having the latent vectors can be processed to provide generative topographic mapping (GTM). The GTM is a gaussian processes-based model, which is used for estimation of a manifold in terms of some properties. That is, certain properties are identified for the objects, such as the properties of molecular objects, and the GTM estimates a manifold in the latent space having objects with those properties once the objects are generated. The GTM can be used in combination with the G2S model in order to find objects with desired properties. The GTM can be implemented in order to build a human-readable 2D map of some manifold, which can be colored by some chosen property. Different properties may have different colors or markings. Accordingly, a selected property can be identified with a defined marking or coloring. The GTM includes N gaussians, and is built on a grid M×M (map), where each Gaussian (G) can be translated from the map (2D) into the manifold (R_d). All Gaussians support the topological property on both 2D and R_d manifolds. In some instances, the protocol can modify the process of GTM building in order to maximize diversity of generated objects and update each point p_ij from the M×M grid using the following algorithm:

$${p}^{′} = \frac{1}{\vert N ( p ) \vert} {\sum }_{{p}_{j} ∈ N} \frac{{p}_{i}}{dist ( p , {p}_{i} )} ;$$

where N(p) are neighbors of point p, “dist” is an Euclidean distance in the latent manifold. The GTM with the proposed update is able to smooth the GTM training process using a previously trained G2S is as follows: 1) Gather a set of objects with corresponding labels (e.g., properties of the objects); 2) Finetune the G2S model using objects from Step 1) if the objects are new for the G2S model (e.g., the objects have not yet been generated by the G2S model in the protocol); 3) Obtain latent vectors of the objects using the encoder; 4) Train the GTM and then translate the latent vectors into a 2D map (e.g., Color 2D map) using the labels (e.g., properties); 5) Select the most promising areas from the 2D map of GTM in terms of objects thereof having the desired properties and then translate these objects of the selected promising area into a G2S latent space; 6) Obtain the selected objects in the G2S latent space using the decoder; 7) Calculate the properties of the generated objects; 8) Update the GTM using new objects and corresponding properties from Step 6) and Repeat Steps 1) through 8), or repeat Steps 2) through 8), if there is no need to repeat Step 1).

In some embodiments, the G2S model can be used for graph-based conditional generation of sequences of objects that have desired properties. The generation of objects with predefined desired properties can be performed using the procedure shown in FIG. 3 and described in connection therewith. First, generative conditions (e.g., desired properties of objects) are concatenated with the latent vectors that were produced by the generator from the sample data. Then, the decoder takes concatenated representation of the objects with the properties and produces SMILES sequences of objects having the desired properties.

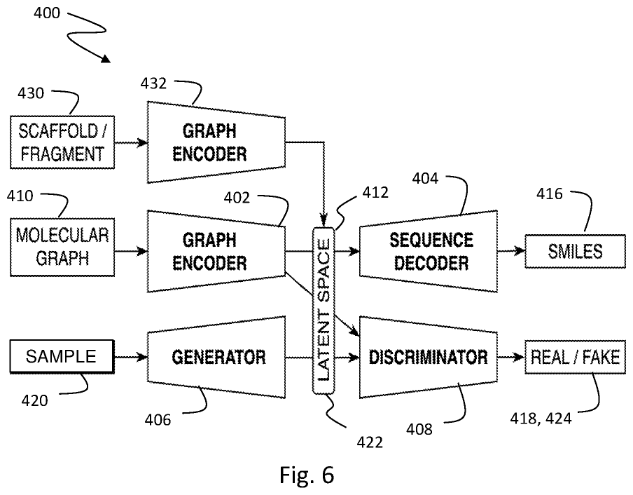

In some embodiments, a DNN, such as the encoder of a G2S model, can be configured to be used for a subgraph conditioning protocol, which protocol can be a condition neural network. It can be a DNN with the same weights as an encoder of G2S or some separate encoder-like GNN with smaller architecture. Given some subgraph (e.g. scaffold or fragment of a molecule) G2S generation process can be conditioned using the output of condition neural network or latent representation of some subgraph. The main goal of such a process is to force the generated graph to contain given subgraph (molecule that contains given scaffold or fragment). An example of graph conditional generation G2S architecture 400 for molecule generation is presented on FIG. 6.

The architecture 400 provides for the graph condition network to condition the data and generation of objects having properties on a continuous subgraph representation using an additional graph encoder neural network 432 (referred to as graph conditional encoder 432). Accordingly, FIG. 6 illustrates an embodiment of a graph-based conditional generation G2S architecture 400, which is shown to include the graph encoder 402, sequence decoder 404, generator 406, and discriminator 408 as well as the additional graph conditional encoder 432. As shown, the graph data 410 (e.g., molecular graph data) is provided to the graph encoder 402, which processes the molecular graph data 410 to obtain the latent space data 412. The architecture 400 also includes the graph conditional encoder 432 being linked to the latent space data 412, where the graph conditional encoder 432 receives conditions data 430, which can be in the form of scaffold or scaffold fragment or structure fragment data (e.g., scaffold data 430). The scaffold data 430 is processed by the graph conditional encoder 432 to generate corresponding latent vectors in the latent space data 412, which can be used similarly to the latent vectors generated by the graph encoder 402. The subgraph conditioning can be done by the graph conditional encoder 432, which can have the same weights as the graph encoder 402. As such, the G2S sequence generation process is conditioned using the output of the conditioned neural network or conditioned latent representation that includes the subgraph conditioning.

The scaffold data 430 can include structural property data for each object. The latent space data 412 from the encoder 402 and graph conditional encoder 432 can be provided to the decoder 404, which is processed to obtain the sequence data 416, such as for example in the form of SMILES data.

The latent space data 412 from the graph encoder 402 and graph conditional encoder 432 can be provided to the discriminator 408, and the discriminator 408 can generate output data 418, such as described herein. The output data 418 can be real output or fake output, which is described more herein. Accordingly, the output data 418 can be sequence data that can correspond with the sequence data of the molecular graph data 410 that was introduced into the graph encoder 402.

Additionally, the architecture 400 includes the generator 406 that is configured for receiving sample data 420. The sample data 420 can be sampled from a natural standard distribution of the object data. The generator 405 can then generate latent space data 422 (e.g., which is different from the latent space data 412 from the graph encoder 402 and the graph data 410 and from the graph conditional encoder 432 and scaffold data 430) in the latent space. The latent space data 422 from the generator 406 can be provided to the discriminator 408, which is processed to obtain the sample output data 424. The sample output data 424 can be real output or fake output, which is described more herein. The architecture 400 can be processed as described herein, such as in connection to FIGS. 1 and 2.

In some embodiments, the outcome of such a process using the architecture 400 is to force the generated sequence data 416 (e.g., whether or not converted to graph data) to contain a given subgraph (e.g., molecule that contains given scaffold or fragment), such as from the scaffold data 430. That is, once the structure of the generated molecule in sequence data is obtained, the structure includes the structure of the given subgraph. For example, the architecture can be used for generating sequence data (e.g., SMILES), such that the structure of the molecules that are generated include the conditioned scaffold data.

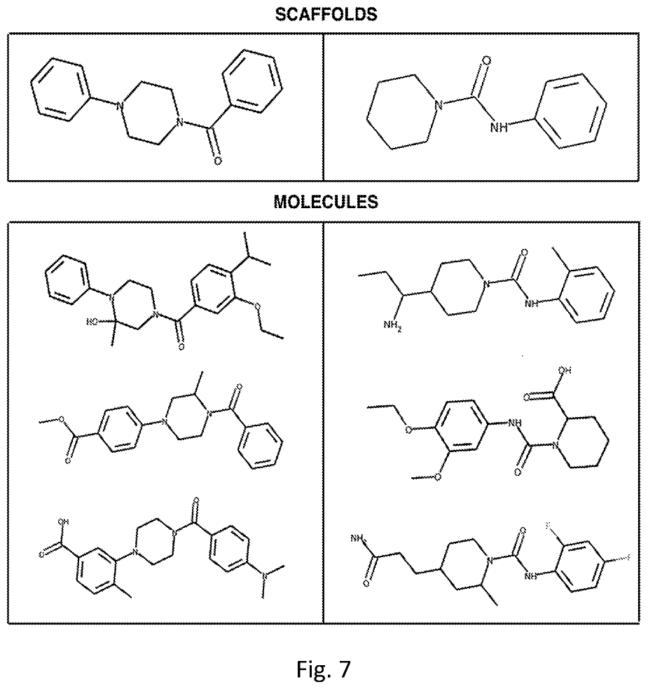

The graph conditioning network allows for the conditioning on continuous subgraph representations by using the additional graph encoder neural network (e.g., 432). The G2S model with the separate graph conditioning network is able to generate molecules with given scaffolds with high accuracy. In the example, the achieved accuracy with regard to specific scaffolds was about 78% accuracy using all unique scaffolds from ZINC250 dataset or with 98% accuracy using one atom type or edge type replacement in generated molecule. The G2S model with the separate graph conditioning network can be able to generate molecules with given fragments (e.g., parts of scaffold or parts of molecules) with 93% accuracy using all unique fragments from ZINC250 dataset or with 100% accuracy using one atom type or edge type replacement in generated molecule. Examples of generated molecules conditioned on given scaffolds is presented on FIG. 7. Accordingly, the architecture 400 is capable of generating molecules that include the scaffold or fragment that is input into the graph conditional encoder 432.

In some embodiments, the architecture described herein can be used in a method for generating new sequences representative of objects from data having graphs, where the new sequences have given (e.g., defined, predetermined) properties (e.g., structure properties or other properties, such as described herein). The method can include providing objects (e.g., in graph data) and their properties (e.g., as conditional data, such as via an additional encoder or concatenation) to a machine learning platform, wherein the machine learning platform outputs a trained model. Then the method includes the machine learning platform takes the trained model and a set of properties for the objects, and outputs new objects with the given properties (e.g., set of properties for the objects). In some aspects, the objects are molecular structures; however, the objects can be pictures, text, sound, or the like. In some aspects, the molecular structures are represented as SMILES strings, InChI, SYBYL line notation (SLN), SMILES arbitrary target specification (SMARTS), Wiswesser line notation (WLN), ROSDAL, or other sequence representations of molecules.

Examples of graph data for molecules can include two or three dimensional adjacency matrix with connections between atoms, atoms and bonds features, molecular adjacency list with atoms and bonds features, COO (coordinate format).

In some aspects, the object properties are biochemical properties of molecular structures of the objects. The biochemical properties can include properties of the molecules that relate to biology, such as receptor activity, binding constant, dissociation constant, epitope binding, or others.

In some aspects, the object properties are structural properties of molecular structures. The structural properties can also be known as physical chemical properties, such as the properties that are used in the field of physical chemistry. Some examples of structural properties can include quantitative estimation of drug likeness (QED), LogP (measure of lipophilicity), SLogP, and other molecular descriptors.

In some embodiments, a model can be generated for the G2S model. The G2S model can include machine learning platform that includes two or more machine learning models. In some aspects, the machine learning platform includes two or more machine learning models and two or more machine learning algorithms. In some aspects, the two or more machine learning models are neural networks such as fully connected neural networks, convolutional neural networks, graph neural networks, recurrent neural networks, or others. In some aspects, the machine learning algorithms include reinforcement learning, Bayesian optimization, or others.

In some embodiments, the machine learning model converts data of a graph object into a latent representation thereof. Then, the machine learning model reconstructs a new object back from the latent codes into a sequence representation of that new object. The machine learning model can enforce a certain distribution of latent codes across all potential objects. The certain distribution thereof can include the desired properties or those properties that are concatenated or processed through the graph conditional encoder.

In some embodiments, the G2S model is trained by adversarial training or variational inference for training thereof.

In some embodiments, the G2S model includes a separate machine learning model that is configured to parameterize the desired distribution of latent codes of objects having the same value of properties. In some aspects, the separate machine learning models are neural network or gaussian processes. In some aspects, the separate machine learning model is a graph neural network and the desired properties are a scaffold or a fragment of a molecular graph.

In some embodiments, the molecular structures that are input into the encoder (e.g., graph encoder) are condensed graphs of reactions, where products are represented as SMIRKS strings. Accordingly, SMILES string is a way to describe a chemical structure in a line of text. Several software packages use SMILES strings as a way to enter and store chemical structure information. A SMIRKS string is a way to describe chemical reactions in text. If you select a reaction and use the Copy As SMILES command, a SMIRKS string is copied to the clipboard. If you use the Paste Special SMILES command when a SMIRKS string is on the clipboard, a reaction is pasted into your document. Accordingly, the object properties can include catalyst properties or type of reaction for the molecule of the object.

While the present G2S model has been described in connection to molecule objects, the models and protocols described herein can be used with objects that are images with descriptions. In some aspects, these descriptions are sequences of natural language words. In some aspects, the properties are images with objects from the original input images.

In some embodiments, the encoder architecture for superior model performance on the selected training sets can include a wide-diagonal convolution architecture. However, the G2S model can also be trained using GNN/GCN-like encoders. These trainings apply to all of the encoders of the described G2S model. Diagonal convolution differs from conventional discrete convolution operation by applying the operation on its diagonal with size n, but not over all input matrices. In this scenario, input matrices are required to be N-gram normalized (e.g., with features that are represented as graph nodes that should be shifted closer to diagonal) before training. For diagonal convolution in a two dimensional situation, the protocols can consider the adjacency matrix A of size N×N, taking a total of n≥1 convolution filters on the first layer of the network. Accordingly, features received after applying filter F at step j can be as follows:

P_{i,j}¹=α( [symbol] F^{1,i},A[j:j+n,j:j+n] [symbol] );

F^{1,i},i∈{1, . . . ,n₀}.

Accordingly, convolutions are applied only on n×n diagonal submatrices. This approach performs well: speedups training and increases overall performance of the model. In a modified G2S, a version of diagonal convolution is used, called wide-diagonal convolution (WDC). The WDC goes not only through the main diagonal, but all diagonals of the input matrix with offset m at each side. More formally features received after applying filter F at step j with vertical (m_v) and horizontal (m_h) offsets is:

P_{i,j}¹=α( [symbol] F^{1,i},A_{[j+m}_{ₕ}_{:j+n+m}_{ₕ}_{,j+m}_{ᵥ}_{:j+n+m}_{ᵥ}_{]} [symbol] );

Accordingly, the WDC is a trade-off between the size of the first layer receptive field and the amount of the parameters to learn.

Additionally, ARAE is often easier to train than AAE for such complex tasks like graph-to-sequence (G2S) mapping, because both the encoder and the generator help each other to find equilibrium. On the other hand, AAE also is used in G2S models. To make the latent space even smoother, Jacobian clamping (JC) regularization for latent space is used in the G2S-ARAE model, such as described herein.

When using the JC, the main goal can be to clamp a Jacobian norm of the generator between two values. In other words, the JC goal is to minimize absolute difference of perturbations between inputs of the generator and outputs of the generator (e.g., produced using these inputs). The JC is a regularization term added to the common model loss. The JC loss is formulated as follows:

Q:=∥G(z)−G(z′)∥/∥z−z′∥;

Lₘₐₓ=(max(Q,λₘₐₓ)−λmax)²;

Lₘᵢₙ=(min(Q,λₘᵢₙ)−λₘᵢₙ)²;

L=Lₘₐₓ+Lₘᵢₙ;

where z is a batch of sample data, z′ is slightly perturbed z, G is generator network, L_max and L_min are hyperparameters. Accordingly, the JC between L_max=3 and L_min=1 leads to better results.

In some embodiments, the G2S model can be trained. In some aspects, before training, the input graph data can be augmented using breadth first search (BFS). The BFS leads to graph data compression near the diagonal of an adjacency matrix, so the diagonal convolutions can be used in order to process input graph data more naturally. Also, it allows to train the model faster with less parameters.

In some training procedures, a final loss of a G2S model is a sum of three losses: autoencoder loss, adversarial loss, and Jacobian clamping loss.

In some embodiments, the Autoencoder loss is a standard negative log likelihood, where L is the length of the sequence, N is a vocabulary size:

$$NLL = - \frac{1}{L * N} {\sum }_{i = 1}^{L} {\sum }_{j = 1}^{N} {y}_{i , j} log ( {p}_{i , j} ) ;$$

In some aspects, the training can use the WGAN-GP algorithm for the generator and discriminator (critic) training with following losses, where P_g are generated objects, P_r are real objects, D is discriminator (critic), GP is gradient penalty and L is weight coefficient of GP term:

L_{critic}=Eₓ_{_{f}}_{˜P}_{_{g}}[D(x_{f})]−Eₓ_{ᵣ}_{˜P}_{ᵣ}[D(xᵣ)]+GP;

GP=λE_{x˜Px}[(∥∇ₓD(x)∥₂−1)²].

In some embodiments, the encoder network trains with a gradient from the decoder and critic (e.g., in case of ARAE) and it final loss is:

L_{encoder}=NLL(xᵣ)L_{critic}(xᵣ).

Convergence decision are made based on reconstruction loss and generation metrics (e.g. Frechet Inception Distance). In the case of the property optimization tasks properties of generated objects are taken into account.

In some embodiments, a method for training a model to generate an object can include an autoencoder step, such as follows: providing a model configured as a graph-to-sequence (G2S) model; obtaining graph data for a plurality of real objects; inputting the graph data into an encoder; generating latent data having latent vectors in a latent space from the graph data with the encoder; obtaining property data of the real objects; concatenating the latent vectors from the graph data with the property data in the latent space; inputting latent space data into a decoder; generating sequence data from the latent space data with the decoder, wherein the sequence data represents real objects and includes symbol logits; computing a log-likelihood between the logits of the sequence data and sequence data of the obtained graph data; inputting latent space data into a discriminator; generating discriminator output data from the discriminator, wherein the discriminator output data includes discriminator logits; computing a log-likelihood of the discriminator logits and labels “1”, wherein labels “1” is a real output data of the discriminator; performing a gradient descent step for the encoder and decoder; and reporting a trained G2S model. The reporting can be via physical report (e.g., paper) or electronic report, which may be displayed on a display screen of a computing system, or the reporting can store the model in a database.

In some embodiments, a method for training a model to generate an object can include a generator step comprising: obtaining sample data of a normal distribution; inputting the sample data into a generator; generating sample latent vectors with the generator, wherein the sample latent vectors are in the latent space; concatenating the property data with the sample latent vectors; inputting latent space data into the discriminator to obtain discriminator sample data having sample logits; computing a log-likelihood of the discriminator sample logits and labels “1”, wherein labels “1” is a real output data of the discriminator; computing a Jacobian clamping term for the generator; performing a gradient descent step for the encoder and decoder; and reporting a generator trained G2S model. The reporting can be via physical report (e.g., paper) or electronic report, which may be displayed on a display screen of a computing system, or the reporting can store the model in a database.

In some embodiments, a method for training a model to generate an object can include a discriminator step comprising: computing a log-likelihood of the discriminator sample logits and labels “0”, wherein labels “0” is a fake output data of the discriminator; performing a gradient descent step for the discriminator using outcome from the log-likelihood of the discriminator logits and labels “1”, and from log-likelihood of the discriminator sample logits and labels “0”; and reporting a generator trained G2S model. The reporting can be via physical report (e.g., paper) or electronic report, which may be displayed on a display screen of a computing system, or the reporting can store the model in a database.

In some embodiments, the method can include: decreasing a learning rate for the autoencoder step; and performing at least one iteration of the autoencoder step, generator step, and discriminator step.

In some embodiments, the method can include: obtaining real object data having sequence data and property data of sequences in the sequence data; and transforming the sequence data into the graph data.

In some embodiments, the method can include performing an optimization protocol to optimize generation of the objects, each object having a predetermined property. In some aspects, the optimization protocol conditions generation of the objects based on the predetermined property, wherein the condition is a real valued vector of the predetermined property directly passed into the latent space of the G2S model.

In some embodiments, the optimization protocol includes a reinforcement learning protocol, comprising: a) inputting sample data for a normal distribution into the generator; b) obtaining sample latent vectors with the generator; c) obtaining generated objects using the decoder; d) calculating properties of the generated objects, the calculated properties having desired properties; e) when the calculated properties of a sub-set of generated objects are sufficiently close to the desired properties, the parameters of the generator and decoder change to provide an improved latent manifold of the latent space, the improved latent manifold having desired objects with the desired properties; f) repeating steps a) through e) until convergence; and g) providing at least one object having the desired properties.

In some embodiments, the desired properties are selected from solubility, lipophilicity, quantitative estimation of drug likeness, Tanimoto similarity with a target molecule, or combinations thereof.

In some embodiments, the optimization protocol includes a Bayesian optimization protocol on the latent space, comprising: a) providing the G2S model; b) obtaining a batch of points from an identified area in the latent space, the identified area having latent vectors of the objects with the desired properties; c) generating objects with the decoder; d) calculating properties of the decoder-generated objects; e) updating the G2S model with batch of points from step b) and calculated properties from step d); f) repeating steps a) through e) until convergence; and g) providing at least one object having the desired properties.

In some embodiments, the methods can include performing a generative topographic mapping protocol that includes: a) obtaining a set of objects having desired properties; b) obtaining latent vectors of the set of objects with the encoder; c) translating the latent vectors of the set of objects into a 2D map with the properties identified on the 2D map; d) selecting at least one region of the 2D map having the desired properties; e) translating the at least one region into a G2S latent space; f) generating objects using the decoder; g) calculating properties of the generated objects; h) updating the 2D map with objects generated by the decoder and with calculated properties from step g); i) repeating steps b) through h) until obtaining at least one object with the desired properties; and j) reporting the at least one object with the desired properties. The reporting can be performed as described herein. In some aspects, the method can include: training the G2S model with the set of objects having the desired properties; and repeating steps b) through h) until obtaining at least one object with the desired properties; and reporting the at least one object with the desired properties.

In some embodiments, the methods may include: obtaining scaffold data, the scaffold data includes structural data for at least a portion of a molecule; inputting the scaffold data into a scaffold encoder; and generating scaffold latent vectors in the latent space, wherein objects generated by the decoder are conditioned on the structural data, and have at a structure of the at least a portion of the molecule.

In some embodiments, the real objects are molecules and the properties of the molecules are biochemical properties and/or structural properties. In some embodiments, the sequence data includes SMILES, InChI, SYBYL line notation (SLN), SMILES arbitrary target specification (SMARTS), Wiswesser line notation (WLN), ROSDAL, or combinations thereof.

In some embodiments, the G2S model includes a machine learning platform, which includes at least two machine learning models that are neural networks selected from the group consisting of fully connected neural networks, convolutional neural networks, graph neural networks, and recurrent neural networks. In some aspects, the machine learning platform includes at least two machine learning algorithms that are a reinforcement learning algorithm and a Bayesian optimization algorithm.

In some embodiments, the methods may include using a separate machine learning model configured to parameterize a desired distribution of latent vectors of objects having a same value of a desired property. The separate machine learning model is a neural network, Gaussian process, or graph neural network, when the graph neural network the desired properties are a molecular scaffold or fragment thereof.

In some embodiments, the graph data includes condensed graphs of chemical reactions and the sequence data generated by the decoder is SMIRKS data, and wherein the object properties are a type of reaction or a catalyst for the type of reaction.

In some embodiments, the real objects are images and the properties are descriptions having sequences of natural language words.

In some embodiments, a method of generating a new object can include: providing a graph-to-sequence (G2S) model, such as described herein; inputting graph data of real objects and properties thereof into the G2S model; training the G2S model with the graph data and property data to obtain a trained G2S model; inputting desired property data of a desired property into the trained G2S model; generating a new object with the desired property with the trained G2S model; and reporting the new object that has the desired property. In some aspects, the method can include: creating a real version of the new object; and validating the new object to have the desired property. In some aspects, the real object is a molecule and the property of the molecule includes biochemical properties and/or structural properties. In some aspects, the real objects are images and the properties are descriptions having sequences of natural language words.

In some embodiments, the methods of generating the new object can include: inputting sample data of a normal distribution into the generator of the G2S mode; conditioning latent vector data in the latent space with at least one desired property of the object; inputting conditioned latent vector data into the decoder; and generating sequence data of a generated object having the at least one desired property. In some aspects, the normal distribution is a normal distribution of real objects having the at least one desired property.

One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

In one embodiment, the present methods can include aspects performed on a computing system. As such, the computing system can include a memory device that has the computer-executable instructions for performing the method. The computer-executable instructions can be part of a computer program product that includes one or more algorithms for performing any of the methods of any of the claims.

In one embodiment, any of the operations, processes, methods, or steps described herein can be implemented as computer-readable instructions stored on a computer-readable medium. The computer-readable instructions can be executed by a processor of a wide range of computing systems from desktop computing systems, portable computing systems, tablet computing systems, hand-held computing systems as well as network elements, base stations, femtocells, and/or any other computing device.

There is little distinction left between hardware and software implementations of aspects of systems; the use of hardware or software is generally (but not always, in that in certain contexts the choice between hardware and software can become significant) a design choice representing cost vs. efficiency tradeoffs. There are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; if flexibility is paramount, the implementer may opt for a mainly software implementation; or, yet again alternatively, the implementer may opt for some combination of hardware, software, and/or firmware.

The foregoing detailed description has set forth various embodiments of the processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a CD, a DVD, a digital tape, a computer memory, etc.; and a transmission type medium such as a digital and/or an analog communication medium (e.g., a fiber optic cable, a waveguide, a wired communications link, a wireless communication link, etc.).

Those skilled in the art will recognize that it is common within the art to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. Those having skill in the art will recognize that a typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control motors (e.g., feedback for sensing position and/or velocity; control motors for moving and/or adjusting components and/or quantities). A typical data processing system may be implemented utilizing any suitable commercially available components, such as those generally found in data computing/communication and/or network computing/communication systems.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

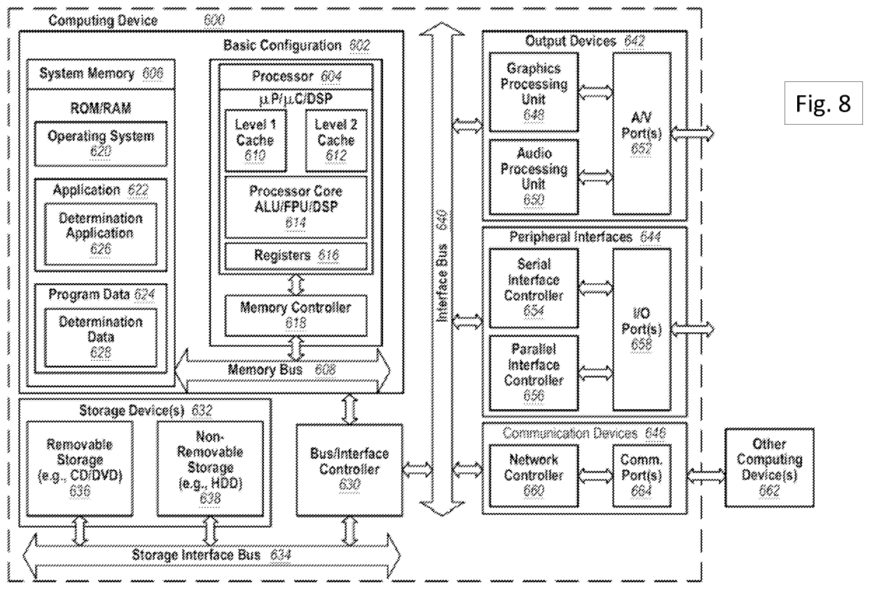

FIG. 8 shows an example computing device 600 that is arranged to perform any of the computing methods described herein. In a very basic configuration 602, computing device 600 generally includes one or more processors 604 and a system memory 606. A memory bus 608 may be used for communicating between processor 604 and system memory 606.

Depending on the desired configuration, processor 604 may be of any type including but not limited to a microprocessor (μP), a microcontroller (μC), a digital signal processor (DSP), or any combination thereof. Processor 604 may include one more levels of caching, such as a level one cache 610 and a level two cache 612, a processor core 614, and registers 616. An example processor core 614 may include an arithmetic logic unit (ALU), a floating point unit (FPU), a digital signal processing core (DSP Core), or any combination thereof. An example memory controller 618 may also be used with processor 604, or in some implementations memory controller 618 may be an internal part of processor 604.

Depending on the desired configuration, system memory 606 may be of any type including but not limited to volatile memory (such as RAM), non-volatile memory (such as ROM, flash memory, etc.) or any combination thereof. System memory 606 may include an operating system 620, one or more applications 622, and program data 624. Application 622 may include a determination application 626 that is arranged to perform the functions as described herein including those described with respect to methods described herein. Program Data 624 may include determination information 628 that may be useful for analyzing the contamination characteristics provided by the sensor unit 240. In some embodiments, application 622 may be arranged to operate with program data 624 on operating system 620 such that the work performed by untrusted computing nodes can be verified as described herein. This described basic configuration 602 is illustrated in FIG. 6 by those components within the inner dashed line.

Computing device 600 may have additional features or functionality, and additional interfaces to facilitate communications between basic configuration 602 and any required devices and interfaces. For example, a bus/interface controller 630 may be used to facilitate communications between basic configuration 602 and one or more data storage devices 632 via a storage interface bus 634. Data storage devices 632 may be removable storage devices 636, non-removable storage devices 638, or a combination thereof. Examples of removable storage and non-removable storage devices include magnetic disk devices such as flexible disk drives and hard-disk drives (HDD), optical disk drives such as compact disk (CD) drives or digital versatile disk (DVD) drives, solid state drives (SSD), and tape drives to name a few. Example computer storage media may include volatile and nonvolatile, removable and non-removable media implemented in any method or technology for storage of information, such as computer readable instructions, data structures, program modules, or other data.

System memory 606, removable storage devices 636 and non-removable storage devices 638 are examples of computer storage media. Computer storage media includes, but is not limited to, RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which may be used to store the desired information and which may be accessed by computing device 600. Any such computer storage media may be part of computing device 600.

Computing device 600 may also include an interface bus 640 for facilitating communication from various interface devices (e.g., output devices 642, peripheral interfaces 644, and communication devices 646) to basic configuration 602 via bus/interface controller 630. Example output devices 642 include a graphics processing unit 648 and an audio processing unit 650, which may be configured to communicate to various external devices such as a display or speakers via one or more A/V ports 652. Example peripheral interfaces 644 include a serial interface controller 654 or a parallel interface controller 656, which may be configured to communicate with external devices such as input devices (e.g., keyboard, mouse, pen, voice input device, touch input device, etc.) or other peripheral devices (e.g., printer, scanner, etc.) via one or more I/O ports 658. An example communication device 646 includes a network controller 660, which may be arranged to facilitate communications with one or more other computing devices 662 over a network communication link via one or more communication ports 664.

The network communication link may be one example of a communication media. Communication media may generally be embodied by computer readable instructions, data structures, program modules, or other data in a modulated data signal, such as a carrier wave or other transport mechanism, and may include any information delivery media. A “modulated data signal” may be a signal that has one or more of its characteristics set or changed in such a manner as to encode information in the signal. By way of example, and not limitation, communication media may include wired media such as a wired network or direct-wired connection, and wireless media such as acoustic, radio frequency (RF), microwave, infrared (IR) and other wireless media. The term computer readable media as used herein may include both storage media and communication media.

Computing device 600 may be implemented as a portion of a small-form factor portable (or mobile) electronic device such as a cell phone, a personal data assistant (PDA), a personal media player device, a wireless web-watch device, a personal headset device, an application specific device, or a hybrid device that include any of the above functions. Computing device 600 may also be implemented as a personal computer including both laptop computer and non-laptop computer configurations. The computing device 600 can also be any type of network computing device. The computing device 600 can also be an automated system as described herein.

The embodiments described herein may include the use of a special purpose or general-purpose computer including various computer hardware or software modules.

Embodiments within the scope of the present invention also include computer-readable media for carrying or having computer-executable instructions or data structures stored thereon. Such computer-readable media can be any available media that can be accessed by a general purpose or special purpose computer. By way of example, and not limitation, such computer-readable media can comprise RAM, ROM, EEPROM, CD-ROM or other optical disk storage, magnetic disk storage or other magnetic storage devices, or any other medium which can be used to carry or store desired program code means in the form of computer-executable instructions or data structures and which can be accessed by a general purpose or special purpose computer. When information is transferred or provided over a network or another communications connection (either hardwired, wireless, or a combination of hardwired or wireless) to a computer, the computer properly views the connection as a computer-readable medium. Thus, any such connection is properly termed a computer-readable medium. Combinations of the above should also be included within the scope of computer-readable media.

Computer-executable instructions comprise, for example, instructions and data which cause a general purpose computer, special purpose computer, or special purpose processing device to perform a certain function or group of functions. Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing the claims.

As used herein, the term “module” or “component” can refer to software objects or routines that execute on the computing system. The different components, modules, engines, and services described herein may be implemented as objects or processes that execute on the computing system (e.g., as separate threads). While the system and methods described herein are preferably implemented in software, implementations in hardware or a combination of software and hardware are also possible and contemplated. In this description, a “computing entity” may be any computing system as previously defined herein, or any module or combination of modulates running on a computing system.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

This patent cross-references: U.S. application Ser. No. 16/015,990 filed Jun. 2, 2018; U.S. application Ser. No. 16/134,624 filed Sep. 18, 2018; U.S. application Ser. No. 16/562,373 filed Sep. 5, 2019; U.S. Application No. 62/727,926 filed Sep. 6, 2018; U.S. Application No. 62/746,771 filed Oct. 17, 2018; and U.S. Application No. 62/809,413 filed Feb. 22, 2019; which applications are incorporated herein by specific reference in their entirety.

All references recited herein are incorporated herein by specific reference in their entirety.

1. A method for training a model to generate an object, the method including an autoencoder step comprising:
providing an variational, adversarial or combination of variational and adversarial autoencoder architecture configured as a graph-to-sequence (G2S) model;
inputting graph data for a plurality of real objects into an encoder of the G2S model;
generating sequence data from latent space data with a decoder of the G2S model;
generating discriminator output data from a discriminator of the G2S model;
performing an optimization for the encoder and decoder; and
reporting a trained G2S model.

2. The method of claim 1, the method including the autoencoder step comprising:
obtaining graph data for a plurality of real objects;
inputting the graph data into an encoder;
generating latent data having latent vectors in a latent space from the graph data with the encoder;
obtaining property data of the real objects;
concatenating the latent vectors from the graph data with the property data in the latent space;
inputting latent space data into a decoder;
generating sequence data from the latent space data with the decoder, wherein the sequence data represents real objects and includes symbol logits;
computing a log-likelihood between the symbol logits of the sequence data and sequence data of the obtained graph data;
inputting latent space data into a discriminator;
generating discriminator output data from the discriminator, wherein the discriminator output data includes discriminator logits;
computing a log-likelihood of the discriminator logits and labels “1”, wherein labels “1” is a real output data of the discriminator;
performing a gradient descent step for the encoder and decoder; and
reporting a trained G2S model.

3. The method of claim 1, further including a generator step comprising:
inputting the sample data of a normal distribution into a generator of the G2S model;
generating discriminator sample data with the discriminator;
performing an optimization for the generator; and
reporting a generator trained G2S model.

4. The method of claim 2, further including a generator step comprising:
obtaining sample samples of a normal distribution;
inputting the sample data into a generator;
generating sample latent vectors with the generator, wherein the sample latent vectors are in the latent space;
concatenating the property data with the sample latent vectors;
inputting latent space data into the discriminator to obtain discriminator sample data having sample logits;
computing a log-likelihood of the discriminator output logits and labels “1”, wherein labels “1” is a real output data of the discriminator;
computing a Jacobian clamping term for the generator;
performing a gradient descent step for the generator; and
reporting a generator trained G2S model.

5. The method of claim 3, further including a discriminator step comprising:
computing an effectiveness of the discriminator;
performing an optimization for the discriminator using the computed effectiveness; and
reporting a discriminator trained G2S model.

6. The method of claim 4, further including a discriminator step comprising:
computing a log-likelihood of the discriminator output logits and labels “0”, wherein labels “0” is a fake output data of the discriminator;
performing a gradient descent step for the discriminator using outcome from the log-likelihood of the discriminator logits and labels “1, and from log-likelihood of the discriminator logits and labels “0; and
reporting a discriminator trained G2S model.

7. The method of claim 5, further comprising:
decreasing a learning rate for the autoencoder step; and
performing at least one iteration of the autoencoder step, generator step, and discriminator step.

8. The method of claim 1, further comprising:
obtaining real object data having sequence data and property data of sequences in the sequence data; and
transforming the sequence data into the graph data.

9. The method of claim 5, further comprising performing an optimization protocol to optimize generation of the objects, each object having a predetermined property.

10. The method of claim 9, wherein the optimization protocol conditions generation of the objects based on the predetermined property, wherein the condition is a real valued vector of the predetermined property directly passed into the latent space of the G2S model.

11. The method of claim 6, further comprising an optimization protocol that includes a reinforcement learning protocol, comprising:
a) inputting sample data for a normal distribution into the generator;
b) obtaining sample latent vectors with the generator;
c) obtaining generated objects using the decoder;
d) calculating properties of the generated objects, the calculated properties having desired properties;
e) when the calculated properties of a sub-set of generated objects are sufficiently close to the desired properties, the parameters of the generator and decoder change to provide an improved latent manifold of the latent space, the improved latent manifold having desired objects with the desired properties;
f) repeating steps a) through e) until convergence; and
g) providing at least one object having the desired properties.

12. The method of claim 11, wherein the desired properties are selected from solubility, lipophilicity, quantitative estimation of drug likeness, Tanimoto similarity with a target molecule, or combinations thereof.

13. The method of claim 6, further comprising an optimization protocol that includes a Bayesian optimization protocol on the latent space, comprising:
a) providing the G2S model;
b) obtaining a batch of points from an identified area in the latent space, the identified area having latent vectors of the objects with the desired properties;
c) generating objects with the decoder;
d) calculating properties of the decoder-generated objects;
e) updating the G2S model with batch of points from step b) and calculated properties from step d);
repeating steps a) through e) until convergence; and
g) providing at least one object having the desired properties.

14. The method of claim 6, further comprising performing a generative topographic mapping protocol, comprising:
a) obtaining a set of objects having desired properties;
b) obtaining latent vectors of the set of objects with the encoder;
c) translating the latent vectors of the set of objects into a 2D map with the properties identified on the 2D map;
d) selecting at least one region of the 2D map having the desired properties;
e) translating the at least one region into a G2S latent space;
f) generating objects using the decoder;
g) calculating properties of the generated objects;
h) updating the 2D map with objects generated by the decoder and with calculated properties from step g);
i) repeating steps b) through h) until obtaining at least one object with the desired properties; and
j) reporting the at least one object with the desired properties.

15. The method of claim 14, further comprising:
training the G2S model with the set of objects having the desired properties; and
repeating steps b) through h) until obtaining at least one object with the desired properties; and
reporting the at least one object with the desired properties.

16. The method of claim 1, further comprising:
obtaining scaffold data, the scaffold data includes structural data for at least a portion of a molecule;
inputting the scaffold data into a scaffold encoder; and
generating scaffold latent vectors in the latent space,
wherein objects generated by the decoder are conditioned on the structural data, and have at a structure of the at least a portion of the molecule.

17. The method of claim 1, wherein the real objects are molecules and the properties of the molecules are biochemical properties and/or structural properties.

18. The method of claim 1, wherein the sequence data includes SMILES, InChI, SYBYL line notation (SLN), SMILES arbitrary target specification (SMARTS), Wiswesser line notation (WLN), ROSDAL, or combinations thereof.

19. The method of claim 1, wherein the G2S model includes a machine learning platform, which includes at least two machine learning models that are neural networks selected from the group consisting of fully connected neural networks, convolutional neural networks, graph neural networks, and recurrent neural networks.

20. The method of claim 19, wherein the machine learning platform includes at least two machine learning algorithms that are a reinforcement learning algorithm and a Bayesian optimization algorithm.

21. The method of claim 5, further comprising a separate machine learning model configured to parameterize a desired distribution of latent vectors of objects having a same value of a desired property, wherein the separate machine learning model is a neural network, Gaussian process, or graph neural network, when the graph neural network the desired properties are a molecular scaffold or fragment thereof.

22. The method of claim 5, wherein the graph data includes condensed graphs of chemical reactions and the sequence data generated by the decoder is SMIRKS data, and wherein the object properties are a type of reaction or a catalyst for the type of reaction.

23. The method of claim 1, wherein the real objects are images and the properties are descriptions having sequences of natural language words.

24. A method of generating an object, the method comprising:
providing a graph-to-sequence (G2S) model;
inputting graph data of real objects and properties thereof into the G2S model;
training the G2S model with the graph data and property data to obtain a trained G2S model;
inputting desired property data of a desired property into the trained G2S model;
generating a new object with the desired property with the trained G2S model; and
reporting the new object that has the desired property.

25. The method of claim 24, further comprising:
creating a real version of the new object; and
validating the new object to have the desired property.

26. The method of claim 25, wherein the real object is a molecule and the property of the molecule includes biochemical properties and/or structural properties.

27. The method of claim 25, wherein the real objects are images and the properties are descriptions having sequences of natural language words.

28. The method of claim 24, comprising:
inputting sample data of a normal distribution into the generator of the G2S mode;
conditioning latent vector data in the latent space with at least one desired property of the object;
inputting conditioned latent vector data into the decoder; and
generating sequence data of a generated object having the at least one desired property.

29. The method of claim 28, wherein the normal distribution is a normal distribution of real objects having the at least one desired property.
